(19) United States
(12) Patent Application Publication
(10) Pub. No.: US 2023/0112563 A1
(43) Pub. Date: Apr. 13, 2023

(54) COMPOSITIONS AND METHODS OF USING MODIFIED LIPOSOMES

(71) Applicant: Siemens Healthcare Diagnostics Inc., Tarrytown, NY (US)

(72) Inventors: Michelle Son, Hopewell Junction, NY (US); Huey Lee, Millwood, NY (US)

(73) Assignee: Siemens Healthcare Diagnostics Inc., Tarrytown, NY (US)

(21) Appl. No.: 17/904,507

(22) PCT Filed: Feb. 1, 2021
(86) PCT No.: PCT/US2021/070104

§ 371 Date: Aug. 18, 2022

Related U.S. Application Data
(60) Provisional application No. 62/985,057, filed on Mar. 4, 2020.

(51) Int. Cl. G01N 33/543

(52) U.S. Cl. CPC G01N 33/5432

(57) ABSTRACT

Disclosed herein are compositions and methods for using modified liposomes comprising (i) an encapsulated hydrophilic acridinium ester (AE), and (ii) a first agent encapsulated by the liposomes and/or (iii) a second agent on the surface of the liposomes. Specifically, the disclosed methods provide methods of labeling a target of interest, assaying a biological sample for a target antigen, and detecting a target antigen in a biological sample. Further disclosed herein are methods for increasing the strength of a signal detected by an imaging modality.

### TECHNICAL FIELD

Disclosed herein are compositions and methods for preparation of liposomes encapsulating hydrophilic acridinium esters and for detecting and labeling a target antigen in a biological sample.

### BACKGROUND

Immunoassay remains the method of choice in the clinical laboratory for analysis of many analytes, particularly complex heterogeneous molecules. A lack of immunoassay signal and sensitivity can be a major obstacle for accurately diagnosing and prognosing a disease. There is a constant need in the art for improved immunoassay labeling methods that provide quick and reliable results which would benefit both patients and healthcare providers.

### SUMMARY

Disclosed herein are modified liposomes comprising (i) an encapsulated hydrophilic acridinium ester (AE), and (ii) a first agent encapsulated by the liposomes and/or (iii) a second agent on the surface of the liposomes.

In some embodiments, the first agent comprises a nucleic acid, a hydrophobic drug, or a hydrophilic drug.

In some embodiments, the second agent comprises a polypeptide, an antibody, a carbohydrate, a polyethylene glycol (PEG), a PEGylated polypeptide, a small molecule, or a drug. In some embodiments, the polypeptide is a biotin, an avidin, a streptavidin, or a fluorescein. In some embodiments, PEGylated polypeptide comprises PEGylated antibody or PEGylated biotin. In other embodiments, the drug is a hydrophobic drug, or a drug conjugated to the surface of the liposome.

According to some embodiments, the encapsulated hydrophilic AE has a concentration ranging from at least 1×10⁻⁸ mol/L to at least 1×10⁻⁸ mol/L. In other embodiments, the encapsulated hydrophilic AE comprises at least 1000 to at least 100,000,000,000 hydrophilic AE molecules.

In further embodiments, the diameter of the liposome is about 200 nm to about 1000 nm. In some embodiments, the diameter of the liposome is about 30 nm to about 100 nm.

Also disclosed herein are methods of labeling a target of interest. The methods comprise conjugating the target to the modified liposomes as disclosed herein.

In one aspect, provided herein are methods of assaying a biological sample for a target antigen. The methods comprise (a) combining, in a medium, the biological sample with the presently disclosed modified liposomes; and, examining the medium for target antigen bound to the modified liposomes.

Also provided herein are methods of detecting a target antigen in a biological sample. The methods comprise (a) contacting the biological sample with the disclosed modified liposomes, wherein the modified liposomes specifically conjugate with the target antigen; (b) imaging a signal produced by the conjugated target antigen; and, (c) detecting the signal produced in step (b), thereby detecting the target antigen.

Further provided herein are methods for increasing the strength of a signal detected by an imaging modality. The methods comprise (a) labeling a target by conjugating the target to the disclosed modified liposomes; (b) imaging a signal produced by the labeled target; and, (c) detecting the signal produced in step (b).

Methods for increasing the sensitivity of an immunoassay are also provided. The methods comprise (a) labeling a target by conjugating the target to the disclosed modified liposomes; (b) imaging a signal produced by the labeled target; and, (c) detecting the signal produced in step (b).

### BRIEF DESCRIPTION OF THE DRAWINGS

The summary, as well as the following detailed description, is further understood when read in conjunction with the appended drawings. For the purpose of illustrating the disclosed devices, systems, and methods, there are shown in the drawings exemplary embodiments of the devices, systems, and methods; however, the devices, systems, and methods are not limited to the specific embodiments disclosed. In the drawings:

FIG. 1 is a diagram illustrating various examples of liposome modification using a variety of materials.

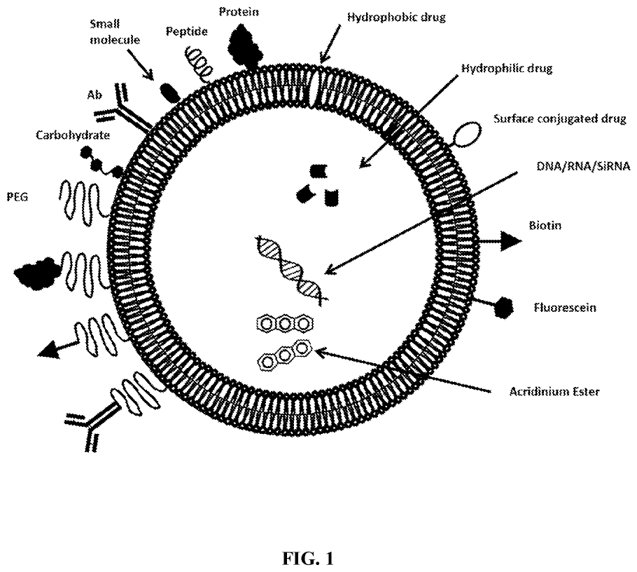

FIGS. 2A-2C are series of diagrams illustrating acridium ester (AE) trapped liposomes. FIGS. 2A and 2B show the liposomes that contain different concentration of AE. The biochemical and biophysical properties of liposome can be modified as shown in FIG. 2C. The surface of liposome is negatively charged in FIG. 2C, while the net charge is neutral in FIGS. 2A and 2B.

FIGS. 3A-3B illustrate that liposomes containing or encapsulating AE (“AE-encapsulating liposomes”) can be made with additional modifications. FIG. 3A: AE-encapsulating liposomes can be further modified with various functional groups including but not limited to biotin, fluorescein, and/or proteins on the liposomal surface. FIG. 3B: The permeability and hydrophilicity of the liposome membranes can be enhanced by adding polyethyleneglycol (PEG) on the surface of the liposome.

FIG. 4 is a series of diagrams showing that the diameter of AE-encapsulating liposomes that trap AE can be generated with different sizes. The AE trapped liposomes with 30 nm, 50 nm, and 100 nm were prepared.

### DETAILED DESCRIPTION OF ILLUSTRATIVE EMBODIMENTS

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only and is not intended to be limiting.

As used herein, the singular forms “a,” “an,” and “the” include the plural referents unless context clearly indicates otherwise. Similarly, the word “or” is intended to include “and” unless the context clearly indicates otherwise.

As used herein, the term “about” will be understood by persons of ordinary skill in the art and will vary to some extent on the context in which it is used. As used herein when referring to a measurable value such as an amount, a concentration, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

The term acridinium ester, as disclosed herein, refers to any acridinium ester which can be encapsulated within a liposome and which can generate a chemiluminescent signal.

The term “analyte” as used herein is a broad term and is used in its ordinary sense, including, without limitation, to refer to a detectable component or target of interest in a sample, such as a substance or chemical constituent in a biological liquid (for example, blood, interstitial liquid, cerebral spinal liquid, lymph liquid or urine). Analytes can include naturally occurring substances, artificial substances, metabolites, and/or reaction products. Examples of analytes include but are not limited to a ligand that is mono- or polyepitopic, antigenic, or haptenic or a nucleic acid such as DNA or RNA.

The term “solid support”, “support structure”, and “substrate” as used herein are used interchangeably and refer to a material or group of materials having a rigid or semi-rigid surface or surfaces. There is no limitation to the shape or size of the support structures. In many embodiments, the solid support(s) will take the form of beads (e.g., silica beads, magnetic beads, paramagnetic beads, and the like), resins, gels, microspheres, or other geometric configurations.

As used herein, a “functional group” refers to a chemical group within a molecule that is responsible for characteristic chemical reactions. Exemplary functional groups include, but are not limited to, those that contain an oxygen, a nitrogen, a phosphorus or a sulfur atom such primary amines, carboxyls, carbonyls, aldehydes, sulfhydryls, hydroxyl groups and esters. As used herein, a functional group is reactive with another group if the two groups can react to form a covalent bond.

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences.

A “crosslinker” refers to a linker that joins two other molecules covalently.

The term “liposome” as used herein refers to an artificially formed vesicle or sac made up of a membrane comprising at least one lipid bilayer. The term is understood to exclude naturally occurring vesicles or other naturally occurring membranous substances isolated from cells or biological samples comprising cells. The terms “vesicle” and “liposome” can be synonymous as used herein in reference to the artificially formed sacs comprising a membrane of at least one lipid bilayer. For example, an artificially formed large unilamellar liposomal vesicle, or “LUV,” is termed a vesicle, but is also referred to as a liposome for purposes of this patent application.

Poly(ethylene glycol), commonly known as PEG, refers to an oligomer of ethylene oxide forming a linear chain. PEG molecules can be either linear or can be branched, wherein each molecule has at least two and generally three or more PEG branches or arms emanating from a central core group.

The term “antibody” refers to an immunoglobulin molecule which is able to specifically bind to a specific epitope on an antigen. Antibodies are typically tetramers of immunoglobulin molecules. The antibodies in the present invention may exist in a variety of forms including, for example, polyclonal antibodies, monoclonal antibodies, intracellular antibodies (“intrabodies”), Fv, Fab and F(ab)2, as well as single chain antibodies (scFv), camelid antibodies and humanized antibodies. As contemplated herein, an antibody conjugated to a quantum dot and support structure may specifically or non-specifically recognize and/or bind to an analyte, such that the analyte can be analyzed qualitatively and quantitatively.

As used herein, the terms “comprising,” “including,” “containing” and “characterized by” are exchangeable, inclusive, open-ended and do not exclude additional, unrecited elements or method steps. Any recitation herein of the term “comprising,” particularly in a description of components of a composition or in a description of elements of a device, is understood to encompass those compositions and methods consisting essentially of and consisting of the recited components or elements.

As used herein, the term “consisting of” excludes any element, step, or ingredient not specified in the claim element.

By “disease” is meant any condition or disorder that damages or interferes with the normal function of a cell, tissue, or organ.

“Detect” refers to identifying the presence, absence or amount of a target (e.g. an analyte to be detected.

An “individual”, “patient” or “subject”, as these terms are used interchangeably herein, includes a member of any animal species including, but are not limited to, birds, humans and other primates, and other mammals including commercially relevant mammals such as cattle, pigs, horses, sheep, cats, and dogs. Preferably, the subject is a human.

As used herein, the terms “treatment” and “treating” refer to an approach for obtaining beneficial or desired results including, but not limited to, therapeutic benefit and/or a prophylactic benefit. For example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease.

By therapeutic benefit is meant eradication or amelioration of the underlying disorder being treated or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

Throughout this disclosure, various aspects of the invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Various terms relating to aspects of the description are used throughout the specification and claims. Such terms are to be given their ordinary meaning in the art unless otherwise indicated. Other specifically defined terms are to be construed in a manner consistent with the definitions provided herein.

### DETAILED DESCRIPTION

Provided herein are modified liposomes comprising (i) encapsulated hydrophilic acridinium ester (AE), and (ii) a first agent encapsulated by the liposome and/or (iii) a second agent on the surface of the liposome.

AEs are stable compounds that provide superior immunoassay performance in the form of increased sensitivity when compared with radioisotopes. The use of AEs can be advantageous for a variety of applications such as labelling ligands or analytes (such as antigens); labelling the specific binding partners of ligands or analytes (such as the corresponding antibodies); or labelling nucleic acids and molecules comprising nucleic acids.

The hydrophilic nature of the AEs used in the disclosed modified liposomes render them suitable for encapsulation within liposomes without leakage through the liposome wall. Detailed description of hydrophilic AEs can be found in the art such as in U.S. Pat. No. 5,656,426 A, the disclosure of which is hereby incorporated by reference in its entirety.

In some embodiments, the concentration of hydrophilic AEs encapsulated by the disclosed liposomes is at least 1.10⁻¹⁰ mol/L to at least 1.10⁻⁹ mol/L, at least 1.10⁻⁹ mol/L to at least 1.10⁻⁸ mol/L, at least 1.10⁻⁸ mol/L to at least 1.10⁻⁷ mol/L, at least 1.10⁻⁷ mol/L to at least 1.10⁻⁶ mol/L, at least 1.10⁻⁶ mol/L to at least 1.10⁻⁵ mol/L, at least 1.10⁻⁵ mol/L to at least 1.10⁻⁴ mol/L, at least 1.10⁻⁴ mol/L to at least 1.10⁻⁵ mol/L, at least 1.10⁻³ mol/L to at least 1.10⁻² mol/L, and at least 1.10⁻² mol/L to at least 1.10⁻¹ mol/L. In other embodiments, the hydrophilic AEs have a concentration ranging from at least 1.10⁻⁸ mol/L to at least 1.10⁻⁶ mol/L.

In some embodiments, the modified liposomes comprise at least 10 to at least 100 hydrophilic AE molecules, at least 100 to at least 1,000 hydrophilic AE molecules, at least 1,000 to at least 10,000 hydrophilic AE molecules, at least 10,000 to at least 100,000 hydrophilic AE molecules, at least 100,000 to at least 1,000,000 hydrophilic AE molecules, at least 1,000,000 to at least 10,000,000 hydrophilic AE molecules, at least 10,000,000 to at least 100,000,000 hydrophilic AE molecules, at least 100,000,000 to at least 1,000,000,000 hydrophilic AE molecules, at least 1,000,000,000 to at least 10,000,000,000 hydrophilic AE molecules, at least 10,000,000,000 to at least 100,000,000,000 hydrophilic AE molecules, and at least 100,000,000,000 to at least 1,000,000,000,000 hydrophilic AE molecules. In other embodiments, the modified liposomes comprise at least 1000 to at least 100,000,000,000 hydrophilic AE molecules.

In further embodiments, the modified liposomes disclosed herein comprise various sizes. In some embodiments, the diameter of the liposome is about 20 nm to about 1000 nm. In some embodiments, the diameter of the liposome is about 20 nm to about 30 nm; about 30 nm to about 40 nm; about 40 nm to about 50 nm; about 50 nm to about 60 nm; about 60 nm to about 70 nm; about 70 nm to about 80 nm; about 80 nm to about 90 nm; about 90 nm to about 100 nm; about 100 nm to about 110 nm; about 110 nm to about 120 nm; about 120 nm to about 130 nm; about 130 nm to about 140 nm; about 140 nm to about 150 nm; about 150 nm to about 160 nm; about 160 nm to about 170 nm; about 170 nm to about 180 nm; about 180 nm to about 190 nm; about 190 nm to about 200 nm; about 200 nm to about 250 nm; about 250 nm to about 300 nm; about 350 nm to about 400 nm; about 400 nm to about 450 nm; about 450 nm to about 500 nm; about 500 nm to about 550 nm; about 550 nm to about 600 nm; about 600 nm to about 650 nm; about 650 nm to about 700 nm; about 700 nm to about 750 nm; about 750 nm to about 800 nm; about 800 nm to about 850 nm; about 850 nm to about 900 nm; about 900 nm to about 950 nm; and about 950 nm to about 1000 nm. In other embodiments, the diameter of the liposome is about 20 nm to about 500 nm. In yet other embodiments, the diameter of the liposome is about 30 nm to about 100 nm.

In some embodiments, the first agent encapsulated by the disclosed modified liposomes may be a nucleic acid, a hydrophobic drug, and a hydrophilic drug. Examples of hydrophobic drugs include, but are not limited to, amphotericin, sily bin, docetaxel, simvastatin, haloperidol and albendazole. Examples of hydrophilic drugs include, hut are not limited to doxorubicin hydrochloride, cytosine-arabinoside, ethinylcytidine, and 5-fluoro-deoxyuridine.

In some embodiments, the second agent of the modified liposomes comprises a polypeptide, an antibody or antigen-binding fragment thereof, an aptamer, an affibody, an affimer, a carbohydrate, a polyethylene glycol (PEG), a PEGylated polypeptide, a small molecule, or a drug. In some embodiments, the polypeptide is a biotin, an avidin or an avidin derivative (e.g., neutravidin), a streptavidin, or a fluorescein. In some embodiments, the PEGylated polypeptide comprises PEGylated antibody or PEGylated biotin. In some embodiments, the drug is a hydrophobic drug or a drug conjugated to the surface of the liposome.

The second agent can be a recombinant, chimeric, genetically engineered, or conjugated protein. In some embodiments, the second agent can be a recombinant antibody or antibody fragment. The second agent can include epitopes, antigens, or other modifications useful for molecularly or immunogenically labelling, expressing, or purifying the second agent. For example, in some embodiments, the second agent can be conjugated to a 6-his tag (6-histidine), a Myc tag, an HA tag, FLAG tag, or similar epitope tag to facilitate isolation, separation, or extraction of the second agent from a reaction mixture comprising the compositions described herein and, for example, a biological sample. The second agent of the modified liposomes described herein can be extracted from the liposomes by use of suitable detergents or membrane disrupting mechanical forces. In this way, the second agent can be separated from the liposomes after a biochemical assay is conducted in order to determine the amount of interferent associated with the second agent.

In some embodiments, the modified liposomes described in this application include multilamellar liposomal vesicles (MLVs), small unilamellar liposomal vesicles (SUVs), large unilamellar liposomal vesicles (LUVs), and giant unilamellar liposomal vesicles (GUVs). In some embodiments, the lipid bilayer can comprise sphingolipids, glycerophospholipids, sterols, and sterol derivatives. Sphingolipids to be used can include sphingomyelin and ceramides containing saturated, monounsaturated, and/or polyunsaturated acyl chains of different lengths. Phospholipids with various headgroup structures can be used, including phosphatidic acid (PA), phosphatidylcholine (PC), phosphatidylethanolamine (PE), phosphatidylglycerol (PG), phosphatidylinositol (PI), cardiolipin, phosphatidylserine (PS) containing saturated, monounsaturated, and/or polyunsaturated acyl chains of different lengths. Sterols and sterol derivatives to be used can include cholesterol, brassicasterol, allocholesterol, cholesterol methyl ether, campestanol, campesterol, cholesteryl acetate, coprostanol, desmosterol, dehydrodesmosterol, dihydrocholesterol, dihydrolanosterol, epicholesterol, lathosterol, lanosterol, sitostanol, sitosterol, stigmasterol, zymostenol, and zymosterol.

Lipids that can be used to form the presently disclosed liposomes can comprise either natural or synthetic sphingolipids, glycerophospholipids, sterols, and sterol derivatives. Sphingolipids can be used include sphingomyelin and ceramides containing saturated, monounsaturated, and/or polyunsaturated acyl chains of different lengths. Phospholipids with various headgroup structures can be used include phosphatidic acid (PA), phosphatidylcholine (PC), phosphatidylethanolamine (PE), phosphatidylglycerol (PG), phosphatidylinositol (PI), cardiolipin, phosphatidylserine (PS) containing saturated, monounsaturated, and/or polyunsaturated acyl chains of different lengths. Sterols and sterol derivatives can be used include but not limited to cholesterol, brassicasterol, allocholesterol, cholesterol methyl ether, campestanol, campesterol, cholesteryl acetate, coprostanol, desmosterol, dehydrodesmosterol, dihydrocholesterol, dihydrolanosterol, epicholesterol, lathosterol, lanosterol, sitostanol, sitosterol, stigmasterol, zymostenol, and zymosterol.

Mixtures of lipids can also be used for the disclosed modified liposomes, including mixtures of sphingolipids, glycerophospholipids, sterols, and sterol derivatives. Sterols and sterol derivatives should not be used alone, i.e. sterols and sterol derivatives should be included in mixtures having sphingolipid- or glycerophospholipid-containing liposomes in a range of from about 0% to about 50% of total lipids. In some embodiments, sphingolipids can comprise porcine brain sphingomyelin, chicken egg sphingomyelin, and bovine milk sphingomyelin. In some embodiments, glycerophospholipids can comprise phospholipids with various headgroup structures such as phosphatidylcholine (PC), phosphatidylethanolamine (PE), phosphatidylglycerol (PG), phosphatidylinositol (PI), cardiolipin, phosphatidylserine (PS) with two saturated acyl chains of different lengths (e.g., 1,2-dipalmitoyl-sn-glycero-3-phosphocholine, 1,2-distearoyl-sn-glycero-3-phosphocholine, 1,2-dipalmitoyl-sn-glycero-3-phosphoethanolamine, 1,2-distearoyl-sn-glycero-3-phosphoethanolamine, 1,2-dipalmitoyl-sn-glycero-3-phospho-(1′-rac-glycerol), 1,2-distearoyl-sn-glycero-3-phospho-(1′-rac-glycerol), 1,2-dipalmitoyl-sn-glycero-3-phospho-(1′-myo-inositol), 1,2-distearoyl-sn-glycero-3-phosphoinositol, 1′,3′-bis[1,2-dipalmitoyl-sn-glycero-3-phospho]-glycerol, 1′,3′-bis[1,2-distearoyl-sn-glycero-3-phospho]-glycerol, 1,2-dipalmitoyl-sn-glycero-3-phospho-L-serine, 1,2-distearoyl-sn-glycero-3-phospho-L-serine), and with one saturated acyl chain of different lengths and one monounsaturated acyl chain of different lengths (e.g.,1-palmitoyl-2-oleoyl-sn-glycero-3-phosphocholine, 1-stearoyl-2-oleoyl-sn-glycero-3-phosphocholine,l-palmitoyl-2-oleoyl-sn-glycero-3-phosphoethanolamine, 1-stearoyl-2-oleoyl-sn-glycero-3- phosphoethanolamine, 1-palmitoyl-2-oleoyl-sn-glycero-3-phospho-(1′-rac-glycerol), 1-stearoyl-2-oleoyl-sn-glycero-3-phospho-(1′-rac-glycerol), 1-palmitoyl-2-oleoyl-sn-glycero-3-phosphoinositol, 1′,3′-bis[1-palmitoyl-2-oleoyl-sn-glycero-3-phospho]-glycerol, 1-palmitoyl-2-oleoyl-sn-glycero-3-phospho-L-serine, 1-stearoyl-2-oleoyl-sn-glycero-3-phospho-L-serine), and with one saturated acyl chain of different lengths and one polyunsaturated acyl chain of different lengths (e.g., 1-palmitoyl linoleoyl-sn-glycero-3-phosphocholine, 1-stearoyl-2-linoleoyl-sn-glycero-3-phosphocholine, 1-palmitoyl-2-linoleoyl-sn-glycero-3-phosphoethanolamine, 1-stearoyl-2-linoleoyl-sn-glycero-3-phosphoethanolamine, 1-palmitoyl-2-linoleoyl-sn-glycero-3-phospho-(1′-rac-glycerol), 1-stearoyl-2-linoleoyl-sn-glycero-3- phospho-(1′-rac-glycerol), 1-palmitoyl linoleoyl-sn-glycero-3-phospho-L-serine, 1-stearoyl-2-linoleoyl-sn-glycero-3-phospho-L-serine). In some embodiments, each fatty acid acyl chain has a number of carbon atoms ranging from about 16 to 20. In other embodiments, each fatty acid acyl chain has 16, 18, or 20 carbon atoms. In some embodiments, the number of double bonds per each fatty acid acyl chain ranges from 0 to 2. Sterols and sterol derivatives for use with sphingolipid- or glycerophospholipid-containing liposomes can comprise cholesterol, dihydrocholesterol, epicholesterol, sitosterol, and lathosterol. Sphingolipids and glycerophospholipids can be used either alone or as a mixture of sphingolipids and glycerophospholipids in the presence of sterols and sterol derivatives to form the modified liposomes described in this application. For example, in some embodiments, the liposomes can comprise 100% porcine brain sphingomyelin or a mixture of porcine brain sphingomyelin and cholesterol. In some non-limiting examples, sterols and sterol derivatives can be in the range of from about 0% to about 50% of total lipids, most preferably about 30%.

The disclosed modified liposomes can comprise modified phospholipids. For example, sphingolipids and glycerophospholipids can be modified with small molecules, polyethylene glycol (PEG), fluorescent molecules, fluorescent PEG, and/or bromine. Sphingolipids and glycerophospholipids, sterols, sterol derivatives, and modified versions of lipids are readily available commercially from various sources, such as Sigma-Aldrich (St. Louis, Mo.); Invitrogen (Carlsbad, Calif.); Avanti Polar Lipids (Alabaster, Ala.); Fisher Scientific (Pittsburgh, Pa.); Steraloids (Newport, R.I.).

Also disclosed herein are methods of labeling a target of interest. The methods comprise conjugating the target to the modified liposomes as disclosed herein.

In some embodiments, the target of interest can be an antigen, a hapten, a DNA probe, a RNA probe, or an antibody. In other embodiments, the target of interest can be present in a biological sample. In some embodiments, the biological sample can be, but is not limited to, whole blood, serum, plasma, urine, saliva, semen, or cerebrospinal fluid.

In one aspect, provided herein are methods of assaying a biological sample for a target antigen. The methods comprise (a) combining, in a medium, the biological sample with the presently disclosed modified liposomes; and examining the medium for target antigen bound to the modified liposomes.

In some embodiments, the assays comprise a biochemical assay such as an immunoassay, a clinical chemistry assay or other medical or diagnostic test. In some embodiments, the assays can comprise a sandwich assay or an in-situ hybridization assay.

In some embodiments, the disclosed modified liposomes can be added to or incubated with reagents for the biochemical assay, other than the biological sample. For example, the modified liposomal compositions can be incubated with a buffer provided as a component of a biochemical assay. Additionally, the modified liposomes can be incubated with a reaction mixture for a biochemical assay which mixture includes one or more reagents for the assay and the biological sample. In some embodiments, the modified liposomal compositions are added in suspension form to the biological sample, the reagent, or the reaction mixture for a biochemical assay. In some embodiments, the modified liposomal compositions are reconstituted from “dry form” in the biological sample, the reagent, or the reaction mixture or in one or more components that contribute to the reaction mixture for the biochemical assay.

Also provided herein are methods of detecting a target antigen in a biological sample. The methods comprise (a) contacting the biological sample with the disclosed modified liposomes, wherein the modified liposomes specifically conjugate with the target antigen; (b) imaging a signal produced by the conjugated target antigen; and, (c) detecting the signal produced in step (b), thereby detecting the target antigen.

Further provided herein are methods for increasing the strength of a signal detected by an imaging modality. The methods comprise (a) labeling a target by conjugating the target to the disclosed modified liposomes; (b) imaging a signal produced by the labeled target; and, (c) detecting the signal produced in step (b).

Methods for increasing the sensitivity of an immunoassay are also provided. The methods comprise (a) labeling a target by conjugating the target to the disclosed modified liposomes; (b) imaging a signal produced by the labeled target; and, (c) detecting the signal produced in step (b).

In some embodiments, the disclosed modified liposomes are ruptured, and the amount of signal generated by the encapsulated hydrophilic AE is measured.

In some embodiments, a peptide and/or a nucleic acid are detected using the disclosed modified liposomes. For instance, a DNA or RNA probe is tagged with a ligand such as a hapten or a biotinylated modified nucleotide. The DNA or RNA probe is allowed to hybridize with complementary DNA or RNA and immobilized on a solid support. The immobilized probe is then reacted with the modified liposomes comprising a receptor for the ligand, such as an antibody or if the probe is biotinylated, avidin. The liposomes are ruptured, and the amount of signal generated by the encapsulated acridinium ester is measured.

Other aspects of the present disclosure comprise determining the presence or amount or detecting the biological activity of a target of interest (e.g. peptide or polypeptide) by means known in the art. These means comprise immunoassay devices and methods which may utilize labeled molecules in various sandwich, competition, or other assay formats. Such assays will develop a signal which is indicative for the presence or absence of the peptide or polypeptide. Moreover, the signal strength can, preferably, be correlated directly or indirectly (e.g. reverse-proportional) to the amount of polypeptide present in a sample. Further suitable methods comprise measuring a physical or chemical property specific for the peptide or polypeptide such as its precise molecular mass or NMR spectrum. These methods comprise for instance biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays (e.g. Siemens' platforms like ADVIA Centaur® XPT, ADVIA Centaur® XP, ADVIA Centaur® CP, IMI ULITE® 1000, IMMULITE® 2000 XPi and Atellica®), enzymatic Cobalt Binding Assay (CBA), and latex agglutination assays.

Specific hybridization can be performed under high stringency conditions or moderate stringency conditions, as appropriate. In a preferred embodiment, the hybridization conditions for specific hybridization are high stringency. Specific hybridization, if present, is then detected using standard methods. If specific hybridization occurs between the nucleic acid probe and a gene in the test sample, the sequence that is present in the nucleic acid probe is also present in the mRNA of the subject. More than one nucleic acid probe can also be used.

### ILLUSTRATIVE EMBODIMENTS

Provided here are illustrative embodiments of the disclosed technology. These embodiments are illustrative only and do not limit the scope of the present disclosure or of the claims attached hereto.
Embodiment 1. A modified liposome comprising (i) an encapsulated hydrophilic acridinium ester (AE), and (ii) a first agent encapsulated by the liposome and/or (iii) a second agent on the surface of the liposome.
Embodiment 2. The modified liposome of embodiment 1, wherein the first agent comprises at least one selected from the group consisting of: a nucleic acid, a hydrophobic drug, and a hydrophilic drug.
Embodiment 3. The modified liposome of embodiment 1, wherein the second agent comprises a polypeptide, an antibody, a carbohydrate, a polyethylene glycol (PEG), a PEGylated polypeptide, a small molecule, or a drug.
Embodiment 4. The modified liposome of embodiment 3, wherein the polypeptide is a biotin, an avidin, a streptavidin, or a fluorescein.
Embodiment 5. The modified liposome of embodiment 3, wherein the PEGylated polypeptide comprises PEGylated antibody or PEGylated biotin.
Embodiment 6. The modified liposome of embodiment 3, wherein the drug is a hydrophobic drug, or a drug conjugated to the surface of the liposome.
Embodiment 7. The modified liposome of embodiment 1, wherein the encapsulated hydrophilic AE has a concentration ranging from at least 1×10⁻⁸ mol/L to at least 1×10⁻⁶ mol/L.
Embodiment 8. The modified liposome of embodiment 1, wherein the encapsulated hydrophilic AE comprises at least 1000 to at least 100,000,000,000 hydrophilic AE molecules.
Embodiment 9. The modified liposome of embodiment 1, wherein the diameter of the liposome is about 20 nm, to about 1000 nm.
Embodiment 10. The modified liposome of embodiment 9, wherein the diameter of the liposome is about 30 nm, to about 100 nm.
Embodiment 11. A method of labeling a target of interest, the method comprising conjugating the target to the modified liposome according to any preceding embodiment.
Embodiment 12. A method of assaying a biological sample for a target antigen, the method comprising:
   a. combining, in a medium, the biological sample with the modified liposome according to any one of embodiments 1 to 11;
   b. examining the medium for target antigen bound to the modified liposome.
Embodiment 13. A method of detecting a target antigen in a biological sample, the method comprising:
   c. contacting the biological sample with the modified liposome according to any one of embodiments 1 to 11, wherein the modified liposome specifically conjugates with the target antigen;
   d. imaging a signal produced by the conjugated target antigen; and,
   e. detecting the signal produced in step (b), thereby detecting the target antigen.
Embodiment 14. A method for increasing the strength of a signal detected by an imaging modality, the method comprising:
   f. labeling a target by conjugating the target to the modified liposome according to any one of embodiments 1 to 11;
   g. imaging a signal produced by the labeled target; and,
   h. detecting the signal produced in step (b).
Embodiment 15. A method for increasing the sensitivity of an immunoassay, the method comprising:
   i. labeling a target by conjugating the target to the modified liposome according to any one of embodiments 1 to 11;
   j. imaging a signal produced by the labeled target; and,
   k. detecting the signal produced in step (b).

### EXAMPLES

The following examples are provided to further describe some of the embodiments disclosed herein. The examples are intended to illustrate, not to limit, the disclosed embodiments.

Materials and Methods

Reagents

1,2-dipalmitoyl-sn-glycero-3-phosphocholine (DPPC); 1-palmitoyl-2-oleoyl-phosphatidylcholine (POPC); 1,2-dioleoyl-sn-glycero-3-phosphocholine (DOPC); 1-palmitoyl-2-oleoyl-sn-glycero-3-phospho-L-serine (POPS); Porcine brain sphingomyelin (SM); cholesterol (CHOL); 1,2-distearoyl-sn-glycero-3-phosphoethanolamine-N-[biotinyl(polyethylene glycol)-2000] (PEG 2000 Biotin-DSPE); 1,2-dipalmitoyl-sn-glycero-3-phosphoethanolamine-N-(biotinyl) (Biotin-DPPE); 1,2-dipalmitoyl-sn-glycero-3-phosphoethanolamine-N-(Rhodamine-DPPE); N-(Fluorescein-5-Thiocarbamoyl)-1,2-Dihexadecanoyl-sn-Glycero-3-Phosphoethanolamine (Fluorescein-DHPE); N-sulfo-propyl-dimethyl acridinium ester-N-hydroxysuccinimide (NSP-DMAE) and Trimethylsilyl propionic DMAE (TSP-DMAE).

Lipids were stored at −20° C. Various polycarbonate membrane filters were used with pore diameter 30, 50, 100, 200, and 400 nanometer.

### Example 1

### Preparation and Purification of Liposomes Encapsulating Acridinium Esters

Provided herein are liposomal vesicles of various sizes (e.g. 30-1000 nm) encapsulating a large number of signal generating molecules (e.g. hundreds of millions) such as acridinium esters (AEs).

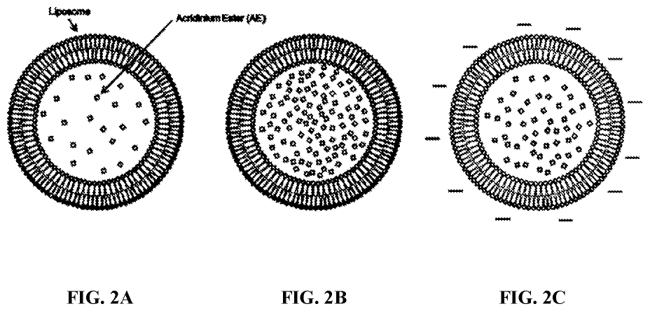

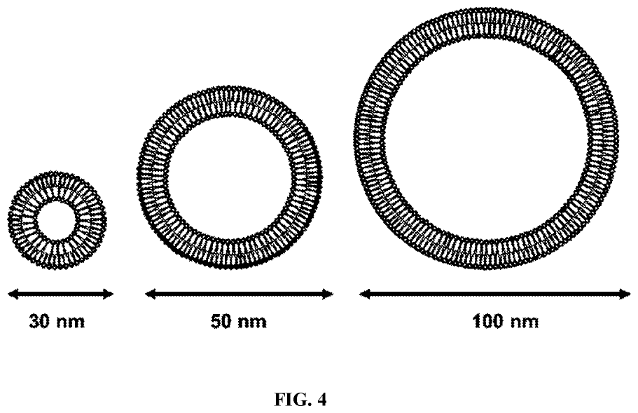

To prepare acridinium ester-encapsulating 4 mM large unilamellar liposomal vesicles (LUVs), lipids (SM or DPPC or POPC or DOPC or SM/POPC 1/1 or DPPC/POPC 1/2 or POPC/POPS 3/1) were mixed and dried under nitrogen followed by high vacuum for at least 2 hours. The amount of cholesterol used in the liposome was varied between 0 and 50 mol % depending on the specific experiment. All the lipid mixtures contained 0.025 mol % of Rhodamine PE to track the final concentration of liposomes. The dried lipid films were dispersed in NSP-DMAE or TSP-DMAE containing phosphate-buffered saline (PBS, 137 mM NaCl, pH 7.4) at 70° C. and then cooled down to room temperature before use. As shown in FIGS. 2A-2C, various acridinium ester (AE)-encapsulating liposomes can be designed either by varying the concentration of AE (FIGS. 2A-2B) or by modifying the biochemical and biophysical properties of the liposome (e.g. negatively charged liposome, FIG. 2C). The concentration of NSP-DMAE and TSP-DMAE was varied between 0 and 15 mg/mL. The lipid mixture was subjected to 10 cycles of freezing/thawing and then extruded through polycarbonate filters with certain pore diameter (e.g., 30 nm, 50 nm, 100 nm, 200 nm, and 400 nm) to obtain uniform liposome size (FIG. 4). NAP-5 (Sephadex G-25) column was used to remove untrapped NSP-DMAE or TSP-DMAE. Dynamic light scattering (DLS) measurements were conducted both before and after NAP-5 column purification. The particle-size distribution of the liposomes obtained showed that the mean diameter of the liposomes was still maintained after the purification step.

### Example 2

### Preparation and Purification of Liposomes Encapsulating Acridinium Esters with Functional Groups

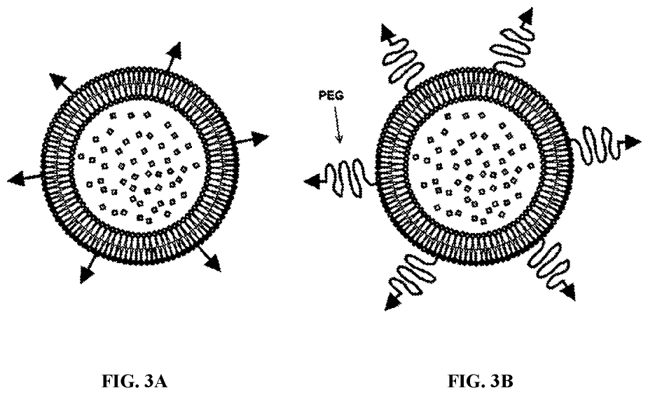

The aim was to define if AE-trapped liposomes can be also prepared efficiently in the presence of various functional groups on the surface of the liposomes and confirm that the addition of functional groups does not impact the resulting liposomes (FIGS. 3A-3B). To do so, Biotin-DPPE, PEG 2000 Biotin-DSPE or Fluorescein-DHPE was added into the lipid mixtures, i.e. SM or DPPC or POPC or DOPC or SM/POPC 1/1 or DPPC/POPC 1/2 or POPC/POPS 3/1 with or without cholesterol as described above, before the lipids were dried under the nitrogen. The amount of Biotin-DPPE, PEG 2000 Biotin-DSPE and Fluorescein-DHPE used in the liposome was varied between 0 and 20 mol %. The permeability and hydrophilicity of the surface of liposomes were particularly enhanced by the addition of polyethyleneglycol (PEG). Dynamic light scattering (DLS) analysis conducted on the resulting liposomes showed that the liposomes can be efficiently formed with 30 nm, 50 nm and 100 nm diameter with functional groups. Addition of Biotin-DPPE, PEG 2000 Biotin-DSPE and Fluorescein-DHPE did not impact the ability of the liposomes to trap acridinium esters. To examine the stability of functional group-containing liposomes, the size of liposomes was maintained throughout.

As shown in FIGS. 3A-3B, the disclosed AE-encapsulating liposomes can include additional modifications. These modifications include, but are not limited to, the addition of various functional groups such as biotin, fluorescein, and/or proteins on the liposomal surface (FIG. 3A). The permeability and hydrophilicity of the liposome membranes can be enhanced by adding polyethyleneglycol (PEG) on the surface of the liposome (FIG. 3B).

Thus, various functional groups including but not limited to biotin, fluorescein, and proteins can be attached to the surface of the disclosed liposomal vesicles. These complexes are useful for detecting a target of interest using immunoassay platforms, such as but not limited to Siemens' platforms like ADVIA Centaur® XPT, ADVIA Centaur® XP, ADVIA Centaur® CP, IMMULITE® 1000, IMMULITE® 2000 XPi and Atellica®. The presently disclosed encapsulated AE vesicles significantly enhance immunoassay signals or relative light units (RLU's) and optimize the output results sensitivity. Depending on the liposomal surface modification, the functionalized AE liposomes can be utilized as a lite reagent or as a signal amplifier.

The disclosures of each and every patent, patent application, and publication cited herein are hereby incorporated herein by reference in their entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A modified liposome comprising (i) an encapsulated hydrophilic acridinium ester (AE), and (ii) a first agent encapsulated by the liposome and/or (iii) a second agent on the surface of the liposome.

2. The modified liposome of claim 1, wherein the first agent comprises at least one selected from the group consisting of: a nucleic acid, a hydrophobic drug, and a hydrophilic drug.

3. The modified liposome of claim 1, wherein the second agent comprises a polypeptide, an antibody, a carbohydrate, a polyethylene glycol (PEG), a PEGylated polypeptide, a small molecule, or a drug.

4. The modified liposome of claim 3, wherein the polypeptide is a biotin, an avidin, a streptavidin, or a fluorescein.

5. The modified liposome of claim 3, wherein the PEGylated polypeptide comprises PEGylated antibody or PEGylated biotin.

6. The modified liposome of claim 3, wherein the drug is a hydrophobic drug, or a drug conjugated to the surface of the liposome.

7. The modified liposome of claim 1, wherein encapsulated hydrophilic AE has a concentration ranging from at least 1×10−8 mol/L to at least 1×10−6 mol/L.

8. The modified liposome of claim 1, wherein the encapsulated hydrophilic AE comprises at least 1000 to at least 100,000,000,000 hydrophilic AE molecules.

9. The modified liposome of claim 1, wherein the diameter of the liposome is about 20 nm, to about 500 nm.

10. The modified liposome of claim 9, wherein the diameter of the liposome is about 30 nm, to about 100 nm.

11. A method of labeling a target of interest, the method comprising conjugating the target to the modified liposome according to any preceding claim.

12. A method of assaying a biological sample for a target antigen, the method comprising:
a. combining, in a medium, the biological sample with the modified liposome according to claim 1;
b. examining the medium for target antigen bound to the modified liposome.

13. A method of detecting a target antigen in a biological sample, the method comprising:
a. contacting the biological sample with the modified liposome according to claim 1, wherein the modified liposome specifically conjugates with the target antigen;
b. imaging a signal produced by the conjugated target antigen; and,
c. detecting the signal produced in step (b), thereby detecting the target antigen.

14. A method for increasing the strength of a signal detected by an imaging modality, the method comprising:
a. labeling a target by conjugating the target to the modified liposome according to claim 1;
b. imaging a signal produced by the labeled target; and,
c. detecting the signal produced in step (b).

15. A method for increasing the sensitivity of an immunoassay, the method comprising:
a. labeling a target by conjugating the target to the modified liposome according to claim 1;
b. imaging a signal produced by the labeled target; and,
c. detecting the signal produced in step (b).
